(19) United States
(12) Patent Application Publication
(10) Pub. No.: US 2025/0109367 A1
(43) Pub. Date: Apr. 3, 2025

(54) Personalized Medicine Platform to Dissect Brain Tumor Microenvironment and Rapidly Test Therapeutic Efficacy

(71) Applicant: University of Cincinnati, Cincinnati, OH (US)

(72) Inventors: Riccardo Barrile, Crescent Springs, KY (US); Kevin Haworth, Cincinnati, OH (US); Soma Sengupta, Chapel Hill, NC (US); Daniel Krummel, Chapel Hill, NC (US)

(73) Assignee: University of Cincinnati, Cincinnati, OH (US)

(21) Appl. No.: 18/902,676

(22) Filed: Sep. 30, 2024

Related U.S. Application Data
(60) Provisional application No. 63/541,038, filed on Sep. 28, 2023.

(51) Int. Cl. C12M 3/00; B33Y 80/00; C12M 1/00; C12M 1/12; C12M 1/34; C12M 1/42; C12M 3/06; C12N 5/071; C12N 5/079; C12N 5/09

(52) U.S. Cl. CPC C12M 21/08; B33Y 80/00; C12M 23/16; C12M 25/14; C12M 29/14; C12M 35/04; C12M 41/36; C12N 5/0622; C12N 5/069; C12N 5/0693

(57) ABSTRACT

A device for modeling a brain tumor microenvironment and testing therapeutic efficacy is provided. The device includes a vascular tissue model and an ultrasound device that is capable of delivering focused ultrasound insonation. The vascular tissue model includes a rigid 3D printed scaffold. The scaffold includes one or more scaffold microfluidic channels, two or more inlets, and a central chamber. The central chamber contains a hydrogel or other biocompatible scaffolds. The hydrogel includes one or more hydrogel microfluidic channels as well as living cells.

### CROSS-REFERENCE TO RELATED APPLICATIONS

This application claims priority to, and the benefit of the filing date of, U.S. Patent Application Ser. No. 63/541,038, filed on Sep. 28, 2023, the disclosure of which is incorporated by reference herein in its entirety

### TECHNICAL FIELD

Aspects of the present invention relate generally to vascular tissue models

### BACKGROUND OF THE INVENTION

This section is intended to introduce the reader to various aspects of the art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

The blood brain barrier (BBB) plays a central role in the function and physiology of the central nervous system (CNS) and is characterized by tight endothelial junctions that protect the brain parenchyma from foreign and immunogenic material to create a privileged environment. The BBB significantly impedes the therapeutic treatment of brain tumors and other neurological disorders. In glioblastoma multiforme (GBM), a highly aggressive brain tumor, a subpopulation of GBM stem cells localizes near microvascular capillaries. These cells subvert cerebrovascular tissue function, transforming the BBB into a brain-tumor barrier (BTB), which shields the tumor microenvironment from therapeutics. Overcoming these transport barriers is critical for the effective delivery of brain-targeted treatments.

An emerging technology to transiently disrupt the BBB and allow passage of therapeutic agents into the CNS is focused ultrasound (FUS). The BBB restricts the penetration of therapeutic agents to the brain. FUS insonation of microbubbles (MB) has enhanced drug delivery via a temporary opening of the tight junctions of the BBB. However, current advances are hindered by reliance on animal models, which limit the exploration of cause and effect due to interspecies differences and the inability to perform high-throughput testing.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

The present invention discloses a device for modeling a brain tumor microenvironment and testing therapeutic efficacy. The device includes a vascular tissue model and an ultrasound device that is capable of delivering focused ultrasound insonation. The vascular tissue model includes a rigid 3D printed scaffold. The scaffold includes one or more scaffold microfluidic channels, two or more inlets, and a central chamber. The central chamber contains a hydrogel or other biocompatible scaffolds. The hydrogel includes one or more hydrogel microfluidic channels. Also, the hydrogel contains living cells.

In one embodiment, the device also includes microbubbles. In another embodiment, the microbubbles are sulfur hexafluoride lipid-type A microspheres. In one embodiment, the hydrogel is either enclosed or open-top.

In another embodiment, the one or more hydrogel microfluidic channels connect to one or more of the scaffold microfluidic channels. In one embodiment, the inlets are capable of connecting to one or more pumps. In another embodiment, the scaffold has an inner surface and the inner surface includes one or more hydrogel anchoring structures. In one embodiment, the rigid 3D printed scaffold is created using stereolithography. In another embodiment, the microfluidic scaffold includes a transparent resin, and further, where the microfluidic scaffold is biocompatible with biological material that may be used in the vascular tissue model.

In one embodiment, the microfluidic scaffold is surface functionalized. In another embodiment, the hydrogel is created using three-dimensional bioprinting. In one embodiment, the hydrogel microfluidic channel has a circular cross section. In another embodiment, the hydrogel is a material selected from the group consisting of fibrin, collagen, matrigel, alginate, gelatin, synthetic polymers, and tissue-specific extracellular matrix.

In one embodiment, the hydrogel comprises stromal cells, brain glioma cells or combinations thereof. In another embodiment, the hydrogel microfluidic channel contains human endothelial cells. In one embodiment, the vascular tissue model is of the human blood-brain barrier.

In another aspect of the invention, a method of modeling a vascular tissue system is disclosed. The method involves inserting a culture with cancer cells in one or more of the hydrogel microfluidic channels of the device described above. Then, the device is connected to one or more pumps. Medium is flowed through the device, including the hydrogel microfluidic channel, while insonating the medium with the ultrasound device. Then data regarding the culture in the hydrogel microfluidic channel is collected.

In one embodiment, the device is perfused with microbubbles. In another embodiment, the culture is a co-culture of human endothelial cells with cancer cells. In one embodiment, the cancer cells are brain glioma cells. In another embodiment, the culture comprises stromal cells and brain glioma cells. In one embodiment, the stromal cells comprise endothelial cells, astroglia cells or combinations thereof.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention. Similar reference numerals are used to indicate similar features throughout the various figures of the drawings:

FIG. 1A is a schematic illustrating the blood brain barrier (BBB).

FIG. 1B is a schematic illustrating enhancing chemotherapeutic delivery with focused ultrasound.

FIG. 2A is a schematic showing an assembled bio-fabricated 3D model.

FIG. 2B is a schematic showing an assembled bio-fabricated 3D model.

FIG. 2C is an image of an industrial 3D printer.

FIG. 2D is an image an assortment of printed scaffolds according to the present invention.

FIG. 2E is an image of a 3D bioprinter.

FIG. 2F is an image of a bioprinter printing a microfluidic vascular channel according to the present invention.

FIG. 2G is a photo of a device according to the present invention.

FIG. 2H is a schematic showing a bio-fabrication method for the 3D model according to the present invention.

FIG. 3 is a schematic showing a model of an assembled bio-fabricated 3D model.

FIG. 4 is a schematic showing an experimental setup according to the present invention.

FIG. 5A is a schematic showing microbubbles passing through the in-vitro bio-fabricated channel.

FIG. 5B is a schematic showing cavitating microbubbles disrupt tight junctions.

FIG. 6 is a graph showing cavitation amplitude vs. peak negative pressure.

FIG. 7A is a series of images showing that FUS causes a significant increase in vascular leakage on-Chip.

FIG. 7B is a graph showing results for Dextran blue. N=3 experiments, *P<0.05; **P<0.01 One-way ANOVA.

FIG. 7C is a graph showing results for BSA. N=3 experiments, *P<0.05; **P<0.01 One-way ANOVA.

FIG. 7D is a pair of images showing image processing and generation of a binary mask highlighting the disruption of cellular junctions (VE-Cadherin) and the formation of gaps in the cellular monolayer after FUS treatment on-chip.

FIG. 8A is a series of representative images of brain endothelial cell morphology and junctions before FUS.

FIG. 8B is a series of representative images of brain endothelial cell morphology and junctions at 2-3 h Post FUS.

FIG. 8C is a series of representative images of brain endothelial cell morphology and junctions at 72 h Post FUS.

FIG. 9A is an image from a first mouse showing that FUS triggered extravasation of Evan's blue dye after FUS, similar to what was observed on-chip.

FIG. 9B is an image from a second mouse showing that FUS triggered extravasation of Evan's blue dye after FUS, similar to what was observed on-chip

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “microbubbles” means lipid or protein-encapsulated gas spheres that are about 1-10 μm in diameter.

### DETAILED DESCRIPTION OF THE INVENTION

One or more specific embodiments of the present invention are described below. In an effort to provide a concise description of these embodiments, all features of an actual implementation may not necessarily be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

In one embodiment, the present invention involves a personalized medicine platform to dissect the brain tumor microenvironment and rapidly test therapeutic efficacy. This invention involves a platform to exploit the power of a microphysiological system (“Organ-on-Chip” technology) and to integrate it with ultrasound technology to facilitate therapeutic delivery to GBM tumors. This integrated platform or ‘Ultrasound-on-Chip’ enables controlled and translational experiments designed to understand and facilitate optimal ultrasound protocols while validating personalized therapies tailored to clearing patients from GBM tumor stem cells typically unable to be targeted by current drugs as well screen therapeutic efficacy for other neurological disorders. The combination of the ability to perform high-throughput screening using microfluidic chip technology and ultrasound technology to disrupt the BBB provides a novel method for personalizing treatment regimens on patient derived samples and improving outcomes in patient treatment.

The terms “microphysiological system” and “Organ-on-Chip” are frequently used as synonyms, both referring to microfluidic-based in vitro models designed to capture the dynamic biochemical microenvironment of living organs. Organ-on-Chip are “called ‘chips’ because they were originally fabricated using methods adapted from those used for manufacturing of computer microchips” such as soft-lithography. The approach of the present invention does not conceive the use of any fabrication method that is related to manufacturing of electronic chips. However, we will use the word “Chip” to refer to the microfluidic chamber used as a rigid microfluidic scaffold

### Blood-Brain Barrier

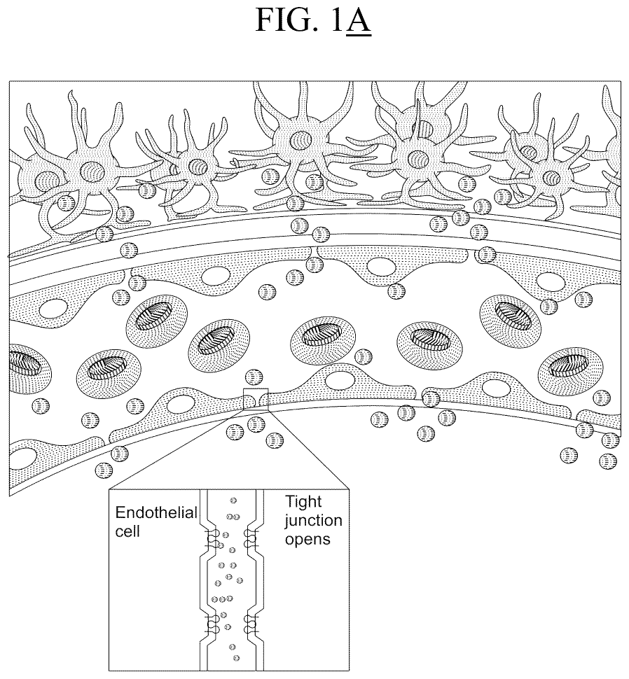

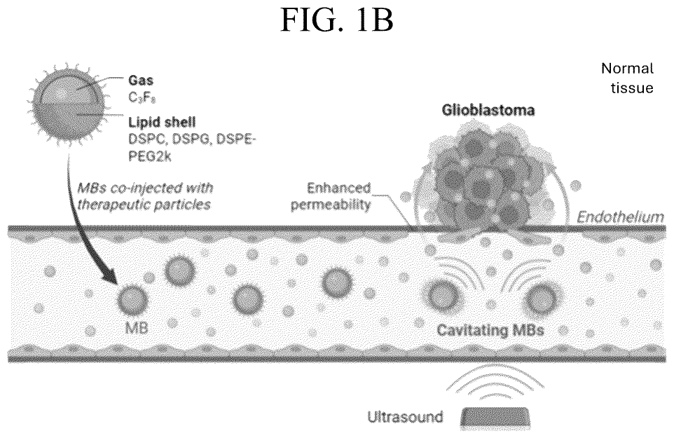

The BBB can be described as a selective permeable border of endothelial cells that prevents solutes in blood from selectively entering extracellular fluid of the central nervous system where neurons reside (see FIG. 1A). The BBB restricts passages of pathogens, large or hydrophilic molecules, and peripheral immune factors (signaling molecules, antibodies, and immune cells). The BBB allows diffusion of hydrophobic molecules (O₂, CO₂, hormones) and small non-polar molecules.

Blood vessels present in the central nervous system (CNS) have specialized properties that facilitate the movement of the certain ions, molecules, and cells between the blood and brain (Blood-brain barrier, BBB). The balance in the CNS homeostasis contributes to neural function, in addition to protecting it from pathogens and toxins. However, the BBB also prevents therapeutics from entering the brain parenchyma, thus severely limiting the ability to treatment options for neurological diseases.

There are several mechanisms by which drugs can cross the blood-brain barrier (BBB). A first mechanism is passive movement of water-soluble agents across the BBB is negligible because of the tight junctions between endothelial cells. A second mechanism is small, lipid-soluble agents, such as antidepressants, cross the BBB via diffusion through endothelial cells. A third mechanism is specialized transport proteins transport glucose, amino acids, and drugs like vinca alkaloids and cyclosporin, across the BBB. A fourth mechanism is receptors mediate transcytosis of proteins like insulin and transferrin. A fifth mechanism is proteins, such as albumin, are adsorbed and transported across the BBB by transcytosis. Finally, a sixth mechanism is efflux transporters counter passive diffusion by pumping foreign agents, such as non-sedating antihistamines, out of the brain.

The BBB is like a regular cell barrier “on steroids.” It is a lipid membrane that separates circulating blood from fluids in the CNS. A good analogy is to think about a child's ball pit: If you throw yourself in, you can get to the bottom—that's like a regular cell membrane. But with the BBB, it's like throwing yourself against a brick wall. The wall is made up of closely packed brain endothelial cells, connected by tight junctions that prevent the passage of molecules and ions.

### Microbubbles

In one embodiment of the present invention, FUS is combined with vascular contrast agents, or microbubbles (MB), to create transient openings in the BBB, facilitating targeted drug delivery to the brain (FIG. 1B). The results presented herein demonstrate that FUS, in the presence of microbubbles, significantly disrupts endothelial cell barrier function both in vitro and in vivo. This disruption is evidenced by increased diffusion of fluorescent dyes, loss of endothelial markers, and formation of gaps in the vascular monolayer, underscoring the potential of FUS to modulate BBB permeability for therapeutic applications. In one embodiment, the microbubbles comprise sulfur hexafluoride lipid-type A microspheres. A commercially available example of such microbubbles is Lumason.

### Biofabricated Models

In one embodiment, the present invention uses focused ultrasound combined with microbubbles to disrupt the tight junctions in a biofabricated 3D model. Biofabricated models are comparable to an animal model. They are faster paced and cheaper, and reliable for disease modeling and drug efficacy testing. In addition, using 3D printing and bio printing makes this model reproducible and compared to traditional 2D model the 3D in-vitro system of the present invention allows us to more accurately mimic the in-vivo tissue environment.

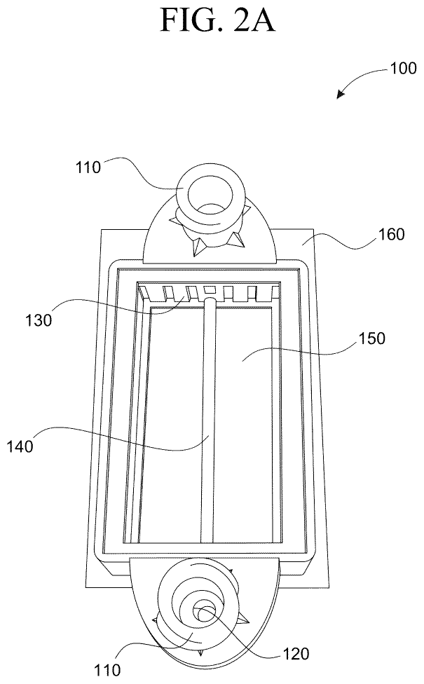

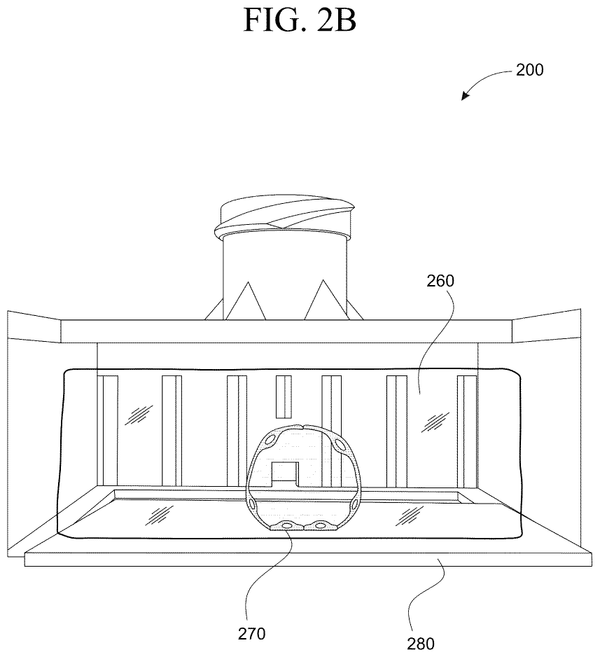

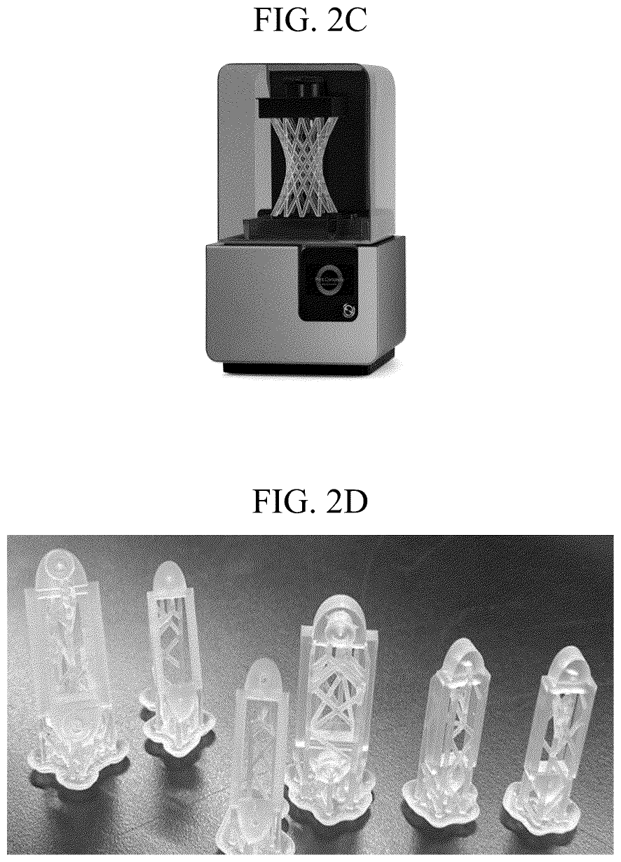

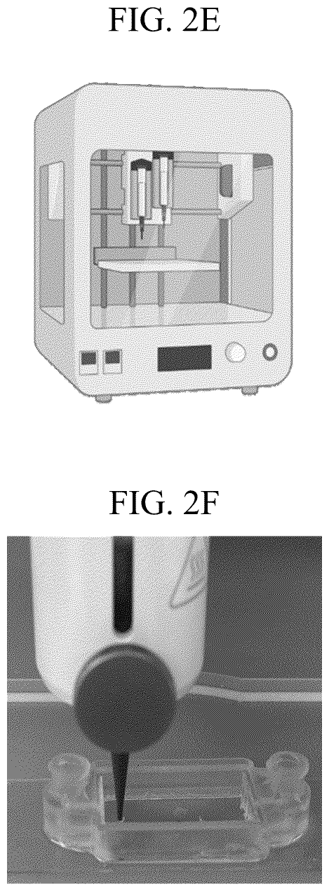

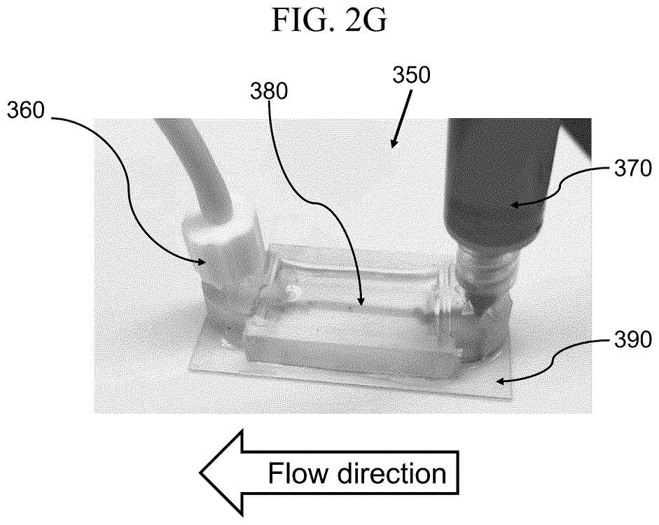

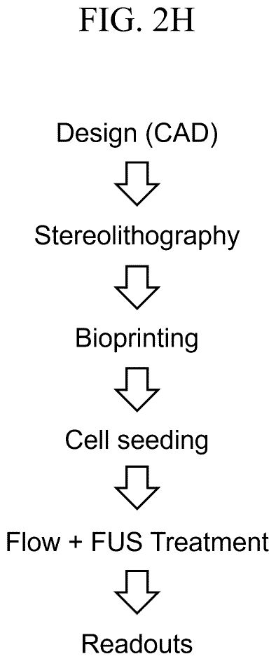

FIGS. 2A-2H show an example of a bio-fabrication method for the 3D model. The combined use of 3D printing and bioprinting represents an enabling approach toward generating scalable and complex vascularized micro physiological systems. The system according to the present invention can be connected with commercially available microfluidic fittings to generate physiologically relevant vascular shear stress. This in vitro platform can be further adapted to conventional analytical readouts including fluorescence microscopy and metabolic assays.

Our innovative approach allows for integrating three-dimensional (3D) cell culture methods with microfluidic principles to generate a perfusable 3D microtissue. In one embodiment, the system consists of one 3D printed microfluidic scaffold obtained via stereolithography (SLA) and used as frame to host a bio-printable cell-laden hydrogel. Referring to FIG. 2A, one or more microfluidic channels connect the rigid scaffold with the hydrogel making this a whole perfusable unit. The 3D printed scaffold 100 incorporates two or more microfluidic ports 110 and inlets 120 designed to facilitate the connection to commercially available pumps. For example, the microfluidic ports 110 located at the top of the device facilitate microfluidic perfusion using commercially available luer-lock fittings. The inner surface of the 3D printed scaffold is lined with hydrogel anchoring structures 130 (or grooves) designed to enable long term (e.g., >10 days) cell culture under fluid flow. The hydrogel can be prepared to encapsulate living cells to mimic the stromal component of living organs. Sacrificial bioinks (such as pluronic or gelatin) may be used to bio-print a perfusive microchannel inside the hydrogel. Importantly, the hydrogel can be made of different biocompatible materials such as fibrin, collagen, Matrigel, alginate, gelatin or synthetic bio-polymers such as PEG or tissue-specific extracellular matrix obtained from human donors or animals in order to faithfully reconstitute the natural 3D microenvironment of a living tissue. The fluidic microchannel 140 embedded into the hydrogel can be seeded with endothelial cells. Before cell seeding, the hollow surface of the hydrogel is coated with tissue-specific extracellular matrix proteins (such as collagen IV) to better reflect the tissue composition of the blood vessels. Ultimately, the use of a 3D hydrogel 150 enables the bio-fabrication of a vascularized synthetic microtissue comprising a perfusable endothelial microchannel surrounded by tissue-specific stromal cells, such as brain astrocytes. The bottom surface of the device 160 is made of transparent glass or vinyl both compatible with conventional microscopes and other optic systems (such as plate readers). The chip's transparency allows researchers to see the organ's functionality, behavior, and response, at the cellular and molecular level.

FIG. 2B provides an alternate view of the central chamber of the device 200, which is designed to host a perfusable hydrogel 260 lined with a hollow microchannel obtained via bio-printing of sacrificial bio-ink. In one embodiment, the hydrogel includes stromal cells such as astrocytes or brain glioma cells. Human endothelial cells may be seeded in the microfluidic channel one or two days after gel polymerization to form a vascular channel. A hydrogel 260 is placed on a transparent glass slide 280. A vascular channel 270 runs through the hydrogel 260.

The combined use of 3D printing and bioprinting represents an enabling approach toward generating scalable and complex vascularized microphysiological systems. Referring to FIG. 2C, an industrial 3D printer (SLA) is used to produce a microfluidic scaffold. FIG. 2D shows a number of printed scaffolds. FIG. 2E shows an image of a 3D bioprinter used to bioprint a microfluidic vascular channel. FIG. 2F is an image of the bioprinter printing such a channel. The system of the present invention can be connected with commercially available microfluidic fittings to generate physiologically relevant vascular shear stress. FIG. 2G is a photo of a system 350 with an outlet 360, a vascularized channel 380, and a transparent surface 390. Medium 370 flows through the vascularized channel 380 in the flow direction indicated in the figure. The system of the present invention can be further adapted to produce conventional analytical readouts for detecting/analyzing, among other things, cell viability, vascular barrier function, immune staining, fluorescence microscopy and metabolic assays. FIG. 2H is a schematic showing a method of using one embodiment of the present invention.

Due to its low cost and high availability, PDMS is the most common material for the fabrication of Organ-on-Chips. However, it is not ideal for acoustic applications, including ultrasounds. PDMS, like other silicon-based materials, absorbs sound waves and poses an impediment to the application of ultrasounds and other acoustic technologies. Recently, we have developed a new approach to generate PDMS-free microfluidic Organ-on-Chip platforms. The innovative approach of the present invention combines commercially available stereolithography 3D printing (SLA) and extrusion bioprinting to generate a perfusable hydrogel harboring living human cells. An overview of this method and design is presented in FIGS. 2A-2H. The bioprinted model, incorporating brain endothelial cells in an engineered blood-vessel-like geometry, is designed to integrate human cell culture with fluidic flow to better mimic the dynamic microenvironment of living tissues. Cell culture medium can be perfused through the endothelialized microchannel. Traditional microscopic techniques, including live imaging, fluorescent, and confocal imaging, can be adapted to obtain qualitative and quantitative information on the status of the vascular wall.

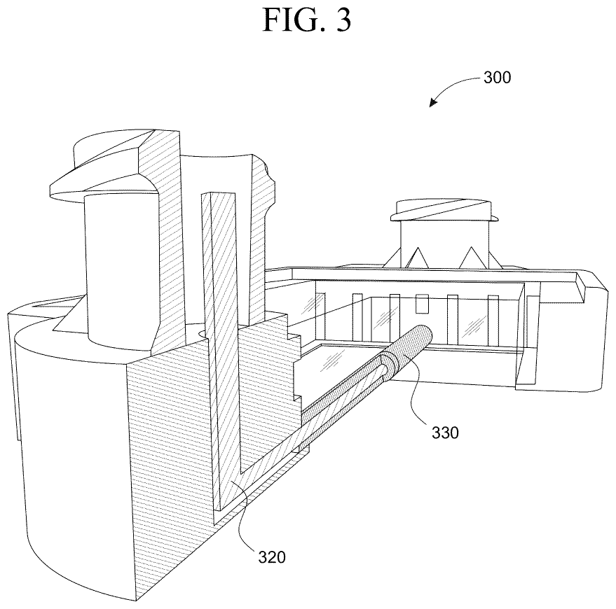

An assembled bio-fabricated 3D model is shown in FIG. 3, which shows that when the device 300 is in use, fluid flow 320 passes inside a vascular wall 330. The 3D bio-model enables culturing of human brain micro-vascular cells (HBMECs) under continuous fluid. The cells were labeled with fluorescent Ve-cadherin. The barrier integrity is determined by using Dextran-Blue 3 kDa.

### Seeding Human Brain Micro Vascular Endothelial Cells (HBMEC)

Prior to seeding the HBMEC cells, the channel was coated with collagen and matrigel, so when the cells were seeded, they did not experience outside stress. Once the cells cover the channel, we refer to it as the vascular wall. In one embodiment, ve-cadherin is used as a junctional protein. It is specific to endothelial cells junctions. It is fluorescently labeled to monitor cell monolayer. A barrier function assay was performed via measuring the amount of fluorescent dye (Dextran-Blue 4 KDa, or Albumin-Red 65 KDa) diffusing outside of the vascular compartment before and after FUS.

### Experimental Setup

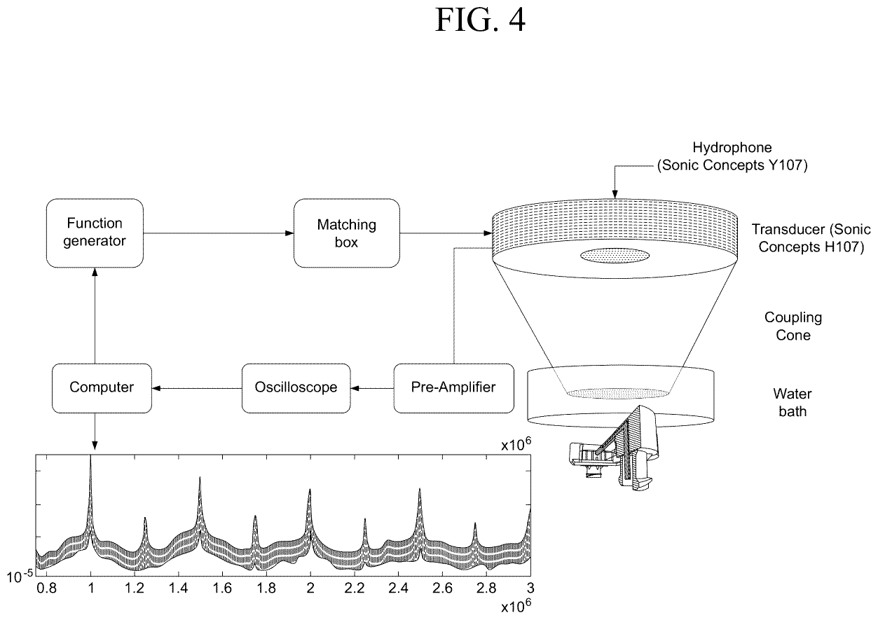

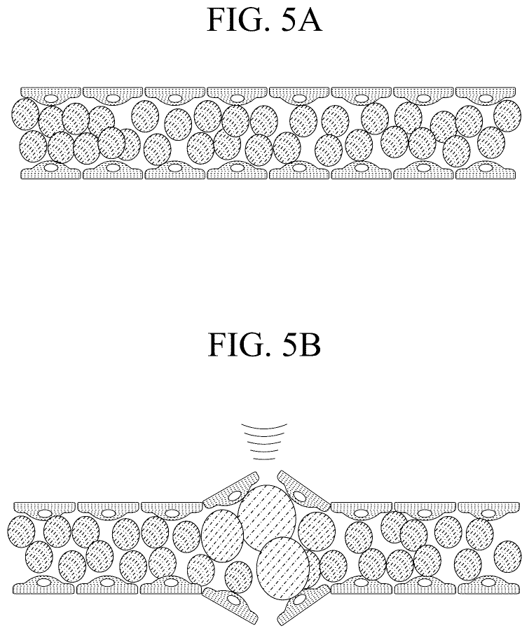

The experimental setup is shown in FIG. 4. Frequency=500 kHz, Pulse repetition frequency=2 HZ, Pulse duration=1 ms, and Amplitude=change to pressure. As media was passed through the 3D model, bubbles would form and disturb the experiment. So, the tubing was prefilled with media. Evans blue dye was combined with microbubbles to track when the bubble would reach the chip. FIG. 5A shows microbubbles passing through the in-vitro bio-fabricated channel. 5B shows how cavitating microbubbles disrupt tight junctions. The model shows microbubbles and Evans blue dye passing through the vascular barrier. Microbubbles get trapped in the acoustic filed and they start oscillating (expanding and shrinking). If this oscillation leads to proper pressure, it can lead to opening of the BBB. Evans blue dye can bind to albumin and pass the blood brain barrier.

### EXAMPLES

### Example 1

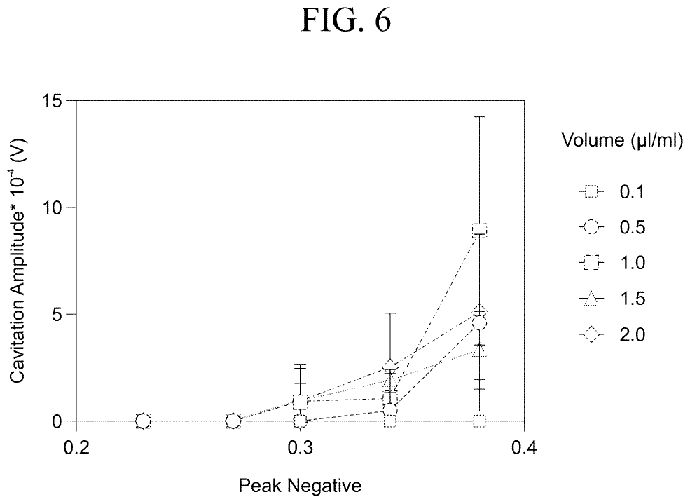

Tests were conducted to determine Lumason concentrations that yield to ultra-harmonic and also, to endothelial cells response to focused ultrasound insonation with microbubbles. In order to optimize the amount of lumason observation of cavitation at the 3rd ultraharmonic was used. Trials were run where microbubble was passed through the channel combined with ultrasound. These experiments were performed based on the volume published in previous in-vivo work. In addition, this experiment also considered the fact that the concentration of the blood depends on how the capillaries are spread. The first volume was equal to 0.03 ml/kg. and through another work it was seen that they have used 50 ul (reference to this work). Studies used 0.38 and 1 ul/ml concentration. FIG. 6 is a graph showing optimization of the microbubbles volume.

### Example 2: Barrier Function Assay Before and After FUS

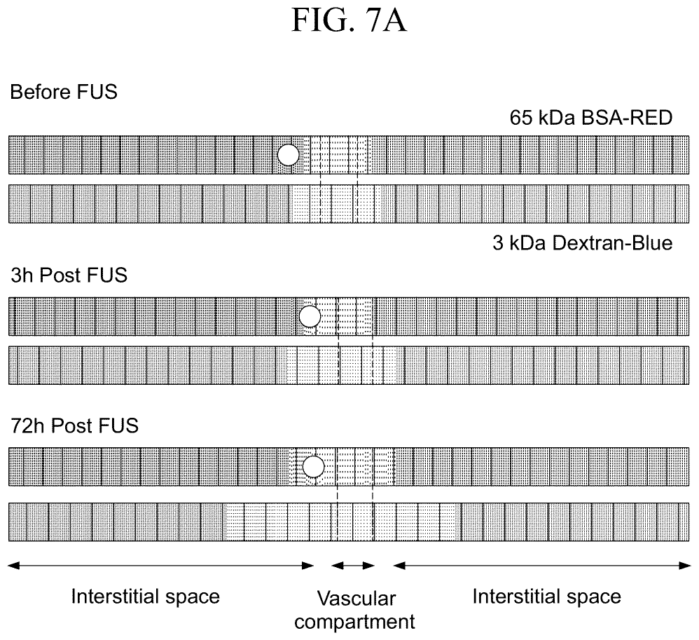

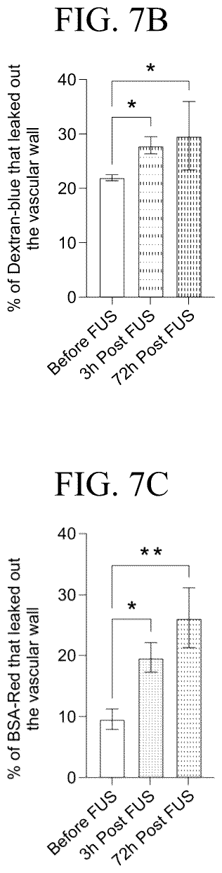

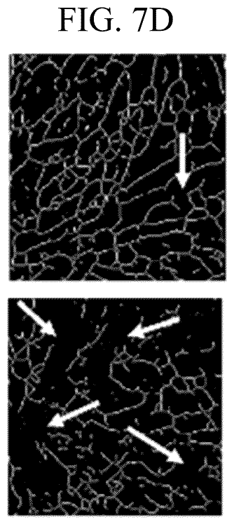

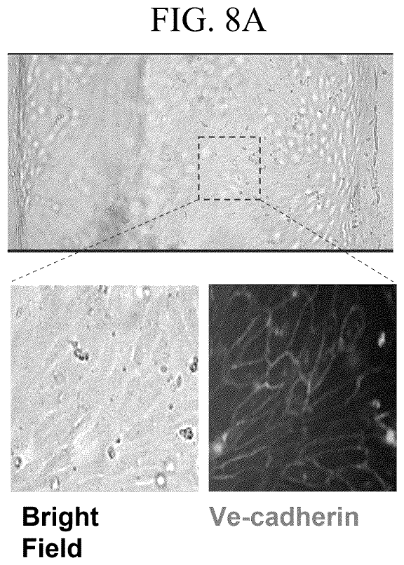

A number of trials were performed regarding barrier function assays (see FIGS. 7A-7C). Prior to each experiment, a barrier assay was conducted to check for the vascular integrity and how tight the endothelial cells are formed. Both dextran blue and BSA were used since they are of different sizes, allowing for the checking of different setups for (for example) new drugs developed, antibodies, etc. Barrier assays were also performed within 3 hours after the treatment with ultrasound. Dextran blue is a more sensitive dye (also smaller). Accordingly, the diffusion of the dye from the vascular wall to the hydrogel is at a higher rate compared to BSA.

Images were taken from the barrier assay in 3 different conditions. After the US treatment, disruption in the junctions results in the leakage of the FTC dextran and BSA outside the vascular integrity. Barrier assay was performed 72 hrs later, and in this case, as seen in the images, the leakage increased. The results show that FUS causes a significant increase in vascular leakage on-Chip. The analysis for the quantification is for one exact point with regard to the center.

### Example 3: Representative Images of Brain Endothelial Cell Morphology and Junctions

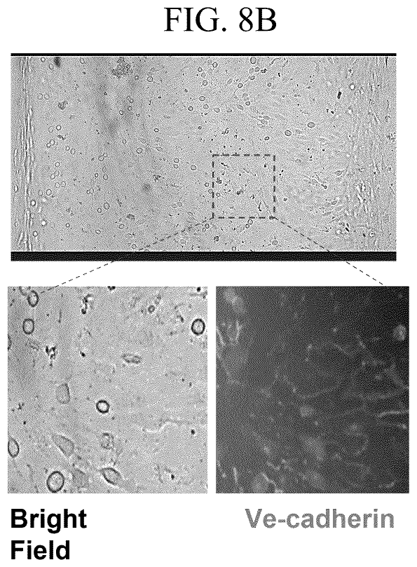

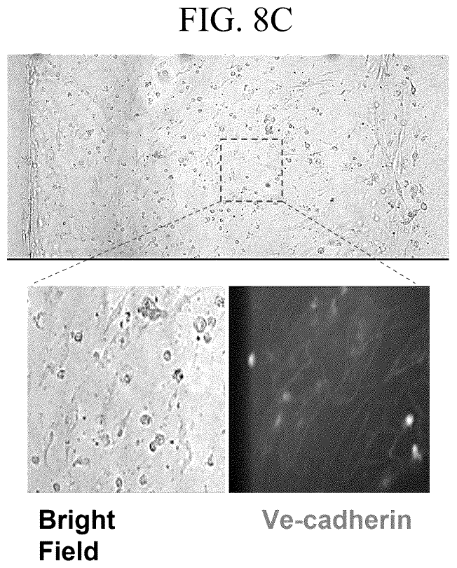

Regarding FIGS. 8A-8C, the images on the top are bright field images. The cells can be seen to be located to the surface of the vascular wall. Cells form a monolayer in the vascular channel. Ve-Cad is specific to endothelial cells. In FIG. 8B, which is taken 2-3 hrs after the ultrasound treatment, we see some changes in the morphology which is predicted to be due the stress from the treatment. And in FIG. 8C the round black dots are dead cells and the round circle with halo interior means, the cells were probably undergoing stress but not dead yet. In the bottom row, the junctions from Ve-cadherin are seen, which is genetically labeled with GFP. 2-3 hr post treatment (FIG. 8B) shows rupture in the junction. In FIG. 8C, the lines are ruptured, however it is not as drastic as what we were seeing in the first part.

### Example 4

The effect of FUS transient disruption was investigated on a human microphysiological model of the BBB consisting of human brain endothelial cells cultured within a 3D-bio printed scaffold designed to reconstitute the architecture of a perfused blood vessel. Cavitation activity was detected in the model when perfused with Lumason microbubbles and exposed to 500 kHz ultrasound at 0.4 MPa for 10 ms pulse duration and 2 Hz pulse repetition frequency. An increase in extravasation of a dye beyond the endothelial layer was detected within 2-3 h of ultrasound insonation. Our results support that this system could be used for targeted BBB opening with a human cell culture model. Moreover, this system holds a promise to further the translational study on US-mediated drug delivery and the development of personalized therapies for patients with brain cancer and other neurovascular disorders. The data indicates that ultrasounds trigger reversible permeability of the cerebrovascular endothelium in a novel 3D-bioprinted model of the human blood-brain barrier.

### Example 5—Focused Ultrasound Triggers Opening of Vascular Wall On-Chip and in Mice

To assess the impact of FUS on the integrity of the vascular wall, a barrier function assay was performed using two fluorescent dyes: Dextran-Blue (4 kDa) and Albumin-Red (65 kDa). These dyes were used to evaluate the diffusion outside the vascular compartment, delineated by red dotted lines in our microfluidic chip model. Due to its smaller size, Dextran-Blue diffused more rapidly compared to Albumin-Red, serving as a more sensitive indicator of barrier integrity. Microscopic imaging of the Chips was used to measure the relative amount of fluorescent dye diffusing out of (extravasing) the vascular channel (FIG. 7A). Image analysis results, represented as the percentage of fluorescent dye detected outside the vascular space, indicated significant diffusion post-FUS. The data, summarized in FIGS. 7B and 7C, show a marked increase in the diffusion of both Dextran-Blue and Albumin-Red following FUS, with statistical significance denoted by *P<0.05 and **P<0.01 (One-way ANOVA, n=6 Chips from N=3 experiments). This suggests a compromise in barrier integrity after FUS treatment. The endothelial cell markers VE-Cadherins and PECAM1 were prominently expressed in the untreated control. However, post-FUS treatment, fluorescent images revealed significant disruption of the vascular monolayer and a notable disappearance of VE-Cadherins.

Further image processing and the generation of a binary mask (FIG. 7D) highlighted the disruption of cellular junctions, evidenced by gaps in the VE-Cadherin-labeled cellular monolayer post-FUS treatment. This disruption corroborates the findings from the barrier function assay, indicating that FUS compromises endothelial cell junction integrity.

### Example 6—In Vivo Validation with Evan's Blue Assay

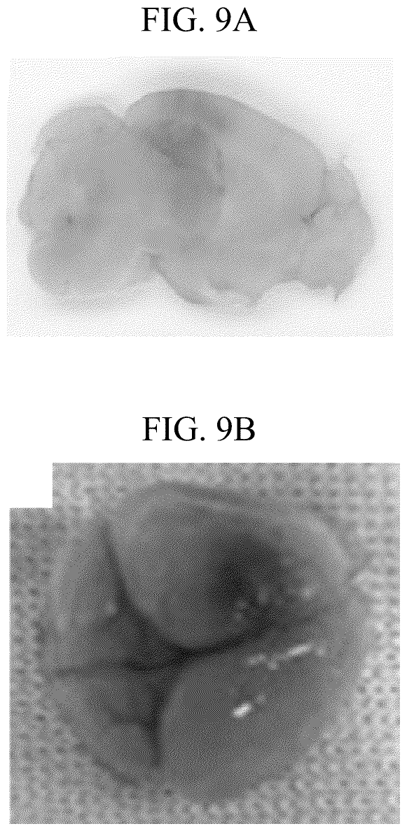

To validate these findings in vivo, a standard Evan's blue assay was conducted in mice under similar conditions. Evan's blue binds to albumin and extravasates into the brain tissue only when the vascular wall is compromised. Results from two mice (FIGS. 9A and 9B) showed extravasation of Evan's blue dye post-FUS, mirroring the on-chip observations. This further confirms that FUS induces a breach in the vascular barrier, consistent with the endothelial response observed in our microfluidic model.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A device for modeling a brain tumor microenvironment and testing therapeutic efficacy comprising a vascular tissue model and an ultrasound device that is capable of delivering focused ultrasound insonation; wherein the vascular tissue model comprises a rigid 3D printed scaffold, the scaffold comprising:
a. one or more scaffold microfluidic channels;
b. two or more inlets; and
c. a central chamber,
wherein the central chamber contains a hydrogel or other biocompatible scaffolds,
wherein the hydrogel comprises one or more hydrogel microfluidic channels, and further, wherein the hydrogel contains living cells.

2. The device of claim 1 further comprising microbubbles.

3. The device of claim 2 wherein the microbubbles comprise sulfur hexafluoride lipid-type A microspheres.

4. The device of claim 1 wherein the hydrogel is either enclosed or open-top.

5. The device of claim 1 wherein the one or more hydrogel microfluidic channels connect to one or more of the scaffold microfluidic channels.

6. The device of claim 1 wherein the inlets are capable of connecting to one or more pumps.

7. The device of claim 1, wherein the scaffold has an inner surface and the inner surface comprises one or more hydrogel anchoring structures.

8. The device of claim 1 wherein the rigid 3D printed scaffold is created using stereolithography.

9. The device of claim 1 wherein the microfluidic scaffold comprises a transparent resin, and further, wherein the microfluidic scaffold is biocompatible with biological material that may be used in the vascular tissue model.

10. The device of claim 1 wherein the microfluidic scaffold is surface functionalized.

11. The device of claim 1 wherein the hydrogel is created using three-dimensional bioprinting.

12. The device of claim 1 wherein the hydrogel microfluidic channel has a circular cross section.

13. The device of claim 1 wherein the hydrogel comprises a material selected from the group consisting of fibrin, collagen, matrigel, alginate, gelatin, synthetic polymers, and tissue-specific extracellular matrix.

14. The device of claim 1 wherein the hydrogel comprises stromal cells, brain glioma cells or combinations thereof.

15. The device of claim 1 wherein the hydrogel microfluidic channel contains human endothelial cells.

16. The device of claim 1 wherein the vascular tissue model is of the human blood-brain barrier.

17. A method of modeling a vascular tissue system comprising:
a. inserting a culture comprising cancer cells in the one or more hydrogel microfluidic channels of the device of claim 1;
b. connecting the device to one or more pumps;
c. flowing medium through the device, including the hydrogel microfluidic channel, while insonating the medium with the ultrasound device; and
d. collecting data regarding the culture in the hydrogel microfluidic channel.

18. The method of claim 17 wherein the device is perfused with microbubbles.

19. The method of claim 17 wherein the culture is a co-culture of human endothelial cells with cancer cells.

20. The method of claim 17 wherein the cancer cells are brain glioma cells.

21. The method of claim 17 wherein the culture comprises stromal cells and brain glioma cells.

22. The method of claim 21 wherein the stromal cells comprise endothelial cells, astroglia cells or combinations thereof.
